(19) United States
(12) Patent Application Publication
(10) Pub. No.: US 2024/0335578 A1
(43) Pub. Date: Oct. 10, 2024

(54) ULTRAVIOLET LIGHT IRRADIATION SYSTEM AND CONTROL METHOD

(71) Applicant: NIPPON TELEGRAPH AND TELEPHONE CORPORATION, Tokyo (JP)

(72) Inventors: Tomohiro TANIGUCHI, Musashino-shi, Tokyo (JP); Ayako IWAKI, Musashino-shi, Tokyo (JP); Satoshi NARIKAWA, Musashino-shi, Tokyo (JP); Takahito KIRIHARA, Musashino-shi, Tokyo (JP); Kazuhide NAKAJIMA, Musashino-shi, Tokyo (JP); Takashi MATSUI, Musashino-shi, Tokyo (JP); Nobutomo HANZAWA, Musashino-shi, Tokyo (JP); Yuto SAGAE, Musashino-shi, Tokyo (JP); Chisato FUKAI, Musashino-shi, Tokyo (JP); Hitoshi SESHIMO, Musashino-shi, Tokyo (JP); Ryuji YAMAMOTO, Musashino-shi, Tokyo (JP); Asuka MIYAKE, Musashino-shi, Tokyo (JP)

(73) Assignee: NIPPON TELEGRAPH AND TELEPHONE CORPORATION, Tokyo (JP)

(21) Appl. No.: 18/574,348

(22) PCT Filed: Jul. 2, 2021
(86) PCT No.: PCT/JP2021/025140

§ 371 Date: Dec. 27, 2023

(51) Int. Cl. A61L 2/10; A61L 2/24; B66B 1/46

(52) U.S. Cl. CPC A61L 2/10; A61L 2/24; A61L 2202/11; A61L 2202/14; B66B 1/46

(57) ABSTRACT

An object of the present invention is to provide an ultraviolet light irradiation system that is easy to operate and can avoid degradation of a material due to ultraviolet light. An ultraviolet light irradiation system according to the present invention includes: one ultraviolet light source unit 11 that generates ultraviolet light; an irradiation unit 13 that irradiates a desired portion (irradiation portion ste) with the ultraviolet light in a limited manner; an optical fiber 14 that propagates the ultraviolet light from the ultraviolet light source unit 11 to the irradiation unit 13; a sensing unit 16 that monitors a state of the irradiation portion ste and outputs the state as monitoring information; and an irradiation control unit 15 that controls output of the ultraviolet light from the ultraviolet light source unit 11 on the basis of the monitoring information.

### TECHNICAL FIELD

The present disclosure relates to an ultraviolet light irradiation system that performs sterilization and virus inactivation by using ultraviolet light, and a control method for the same.

### BACKGROUND ART

For the purpose of preventing infectious diseases and the like, there is an increasing demand for a system that performs sterilization and virus inactivation using ultraviolet light. In particular, a technique is disclosed for performing sterilization and virus inactivation by ultraviolet irradiation on a button or the like of an elevator used by an unspecified number of people (see, for example, Non Patent Literatures 1 and 2.). Note that, in the present specification, the term “sterilization or the like” means sterilization and virus inactivation.

### (1) Mobile Sterilization Robot

Non Patent Literature 1 describes a technique in which a robot equipped with an ultraviolet light irradiation system gets on an elevator and irradiates a target portion for sterilization or the like such as an elevator button with ultraviolet light.

### (2) Stationary System

Non Patent Literature 2 describes a technique in which an irradiation light of ultraviolet light is installed on a ceiling or the like and irradiates a target portion for sterilization or the like such as an elevator button with ultraviolet light.

### CITATION LIST

### Non Patent Literature

Non Patent Literature 1: Nikkei web site (https://www.nikkei.com/article/DGXZQODZ038JA0T01C20A200000 0/), searched on May 26, 2021
Non Patent Literature 2: Emiya Holdings Co., Ltd. Website (https://ene-save.jp/news/5382.html), searched on May 26, 2021

### SUMMARY OF INVENTION

### Technical Problem

However, devices described in Non Patent Literatures have the following problems.

### (A) Operational Difficulty

In the technique of Non Patent Literature 1, it is necessary to move the robot into an elevator or the like each time ultraviolet light irradiation is performed, but it is difficult to perform frequent sterilization or the like in consideration of operation of the elevator or the like. That is, the technique of Non Patent Literature 1 has a problem that the frequency of use of the robot decreases depending on a target of sterilization or the like, and as a result, it is difficult to obtain an effect of sterilization or the like.

### (B) Material Degradation

The irradiation light described in Non Patent Literature 2 has a wide irradiation range of ultraviolet light, and a portion that does not originally need sterilization or the like is also irradiated with the ultraviolet light. Thus, there is a possibility that degradation of a material of the portion is caused by the ultraviolet light. That is, the technique of Non Patent Literature 2 has a problem that it is difficult to avoid degradation of a material due to the ultraviolet light.

To solve these problems, an object of the present invention is to provide an ultraviolet light irradiation system and a control method that are easy to operate and can avoid degradation of a material due to ultraviolet light.

### Solution to Problem

To achieve the above object, in an ultraviolet light irradiation system according to the present invention, ultraviolet light from a light source is transmitted by an optical fiber, is delivered to an irradiation unit disposed inside an elevator or the like, and irradiates a target portion after a beam and an irradiation position are adjusted in the irradiation unit. In addition, an irradiation timing is controlled in cooperation with a sensing function such as a monitoring camera.

Specifically, an ultraviolet light irradiation system according to the present invention includes:
one ultraviolet light source unit that generates ultraviolet light;
an irradiation unit that irradiates a desired portion with the ultraviolet light in a limited manner;
an optical fiber that propagates the ultraviolet light from the ultraviolet light source unit to the irradiation unit;
a sensing unit that monitors a state of the desired portion and outputs the state as monitoring information;
and an irradiation control unit that controls output of the ultraviolet light from the ultraviolet light source unit on the basis of the monitoring information.

In addition, a control method according to the present invention is control method for an ultraviolet light irradiation system including: one ultraviolet light source unit that generates ultraviolet light; an irradiation unit that irradiates a desired portion with the ultraviolet light in a limited manner; an optical fiber that propagates the ultraviolet light from the ultraviolet light source unit to the irradiation unit; and a sensing unit that monitors a state of the desired portion,
the control method including:
outputting the state of the desired portion as monitoring information by the sensing unit; and
controlling output of the ultraviolet light from the ultraviolet light source unit on the basis of the monitoring information.

The present ultraviolet light irradiation system achieves a state in which a target of sterilization or the like can be irradiated with the ultraviolet light at any time with a simple configuration in which only the optical fiber and the irradiation unit are installed in an elevator or the like instead of a robot. The present ultraviolet light irradiation system further includes the sensing unit, and can immediately perform irradiation with the ultraviolet light at a desired time or place to obtain an effect of sterilization or the like, so that operation is easy.

In addition, the present ultraviolet light irradiation system uses the light source such as a laser/LED capable of narrowing the beam, and can irradiate only a target portion for sterilization or the like with the ultraviolet light with pinpoint accuracy. With this configuration, the present ultraviolet light irradiation system can avoid irradiation of an unnecessary portion with the ultraviolet light, and can prevent material degradation of the portion.

Thus, the present invention can provide an ultraviolet light irradiation system that is easy to operate and can avoid degradation of a material due to ultraviolet light.

In addition, the monitoring information of the ultraviolet light irradiation system according to the present invention is information indicating that the desired portion is touched by a person.

The sensing unit detects that a person touches a button of an elevator or the like, and transmits the information to the irradiation control unit as the monitoring information. In addition, the sensing unit detects that a person disappears from the elevator or the like, and also transmits the information to the irradiation control unit. On the basis of these pieces of information, the irradiation control unit can cause the ultraviolet light source unit to irradiate the button touched by a person with the ultraviolet light when the person disappears from the elevator or the like.

In addition, the irradiation unit of the ultraviolet light irradiation system according to the present invention includes an adjustment unit that adjusts an irradiation direction of the ultraviolet light, the monitoring information also includes information on a position of the desired portion touched by a person, and the irradiation control unit instructs the adjustment unit on the irradiation direction of the ultraviolet light on the basis of the monitoring information.

The sensing unit specifies a button of an elevator or the like touched by a person, and transmits the information to the irradiation control unit as the monitoring information. The irradiation control unit causes the adjustment unit of the irradiation unit to change the irradiation direction of the ultraviolet light so that the button is irradiated with the ultraviolet light from the information. Then, the irradiation control unit irradiates the button touched by the person with the ultraviolet light when the person disappears from the elevator or the like.

In the present ultraviolet light irradiation system, the sensing unit such as a monitoring camera monitors the inside of an elevator or the like, so that it is possible to determine a time or a place in which sterilization or the like is required, such as a timing when a person touches a certain button, and it is possible to perform irradiation with the ultraviolet light only at the time or the place.

In addition, the irradiation unit of the ultraviolet light irradiation system according to the present invention includes a plurality of the irradiation units, and an optical branching device that splits the ultraviolet light to each of the irradiation units is further included.

The present ultraviolet light irradiation system has a system configuration in which a single ultraviolet light source unit and a plurality of irradiation units are connected to each other by optical fibers via a distribution function unit, the plurality of irradiation units being respectively installed near a plurality of target portions where sterilization or the like is performed. With this configuration, the present ultraviolet light irradiation system can share a single ultraviolet light source unit in work of sterilization or the like with respect to the plurality of target portions. Thus, the present ultraviolet light irradiation system is economical.

The optical fiber of the ultraviolet light irradiation system according to the present invention is any one of a solid core optical fiber, a hole assisted optical fiber, a hole structure optical fiber, a hollow core optical fiber, a coupling core type optical fiber, a solid core type multi-core optical fiber, a hole assisted type multi-core optical fiber, a hole structure type multi-core optical fiber, a hollow core type multi-core optical fiber, and a coupling core type multi-core optical fiber. The optical fiber can increase a transmission light intensity of the ultraviolet light and reduce leakage loss in a bent portion or the like.

Note that the above inventions can be combined as much as possible.

### Advantageous Effects of Invention

The present invention can provide an ultraviolet light irradiation system that is easy to operate and can avoid degradation of a material due to ultraviolet light.

### BRIEF DESCRIPTION OF DRAWINGS

FIG. 1 is a diagram illustrating an ultraviolet light irradiation system according to the present invention.

FIG. 2 is a diagram illustrating an ultraviolet light irradiation system according to the present invention.

FIG. 3 is a diagram illustrating an ultraviolet light irradiation system according to the present invention.

FIG. 4 is a diagram illustrating a cross-sectional structure of an optical fiber.

FIG. 5 is a diagram illustrating pinpoint irradiation.

FIG. 6 is a diagram illustrating a control method according to the present invention.

### DESCRIPTION OF EMBODIMENTS

Embodiments of the present invention will be described with reference to the accompanying drawings. The embodiments described below are an examples of the present invention, and the present invention is not limited to the following embodiments. Note that components having the same reference numerals in the present specification and the drawings indicate the same components.

### First Embodiment

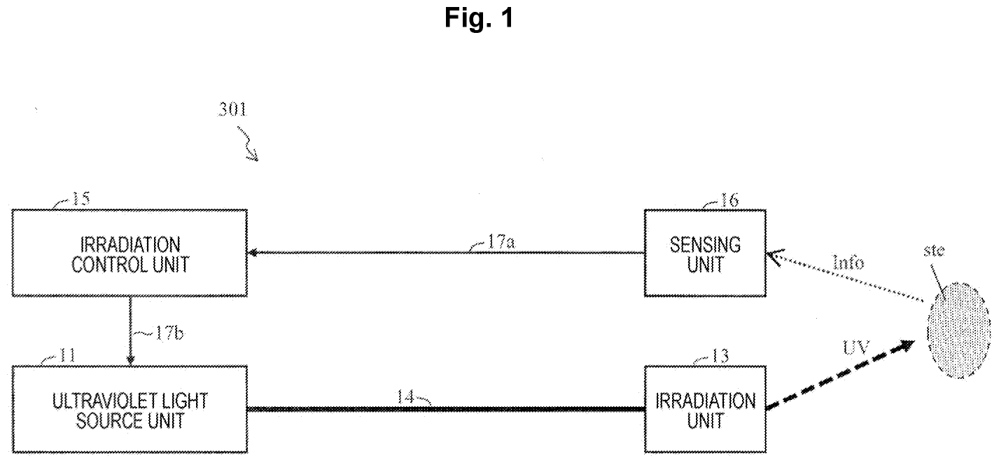

FIG. 1 is a diagram illustrating an ultraviolet light irradiation system 301 of the present embodiment.

The ultraviolet light irradiation system 301
includes:
one ultraviolet light source unit 11 that generates ultraviolet light;
an irradiation unit 13 that irradiates a desired portion (irradiation portion ste) with the ultraviolet light in a limited manner;
an optical fiber 14 that propagates the ultraviolet light from the ultraviolet light source unit 11 to the irradiation unit 13;
a sensing unit 16 that monitors a state of the irradiation portion ste and outputs the state as monitoring information; and
an irradiation control unit 15 that controls output of the ultraviolet light from the ultraviolet light source unit 11 on the basis of the monitoring information.

The ultraviolet light source unit 11 outputs light in an ultraviolet region effective for sterilization. The ultraviolet light source unit 11 outputs the ultraviolet light to the optical fiber 14. In the ultraviolet light source unit 11, power, output, or stop of the ultraviolet light is controlled in accordance with an instruction 17b from the irradiation control unit 15. The ultraviolet light source unit 11 is a light source capable of narrowing a beam of the ultraviolet light emitted from the irradiation unit 13. The ultraviolet light source unit 11 is, for example, a semiconductor laser, a fiber laser, an excimer laser, or a light emitting diode (LED).

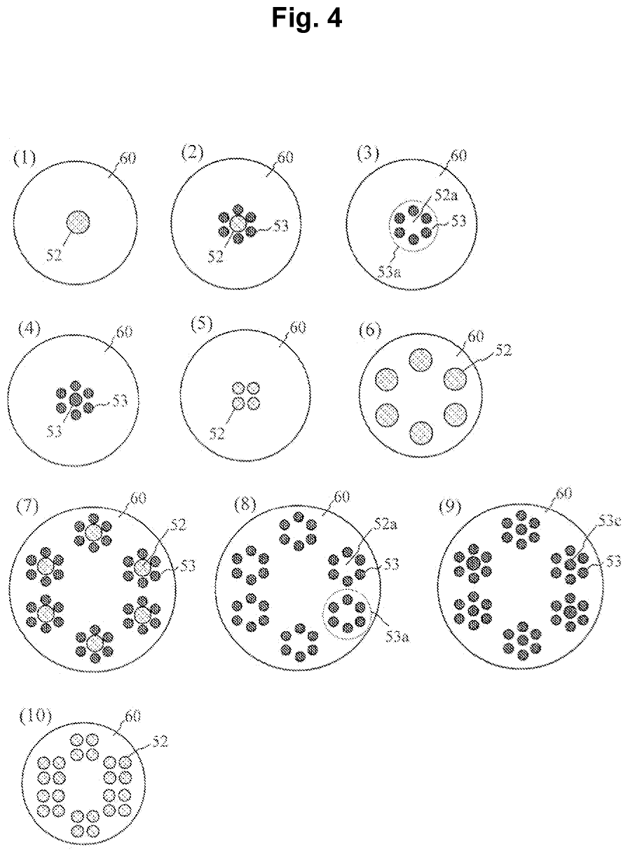

The optical fiber 14 propagates the ultraviolet light from the ultraviolet light source unit 11 to the irradiation unit 13. The optical fiber 14 is preferably an optical fiber having a cross-sectional structure illustrated in FIG. 4, that can propagate high-intensity ultraviolet light and perform sterilization or the like in a short time.

### (1) Solid Core Optical Fiber

This optical fiber has one solid core 52 having a refractive index higher than that of a clad 60, in the clad 60. As used herein, the term “solid” means “not hollow”. Note that, the solid core can also be made by forming an annular low refractive index region in the clad.

### (2) Hole Assisted Optical Fiber

This optical fiber has the solid core 52 and a plurality of holes 53 arranged on the outer periphery thereof, in the clad 60. The medium of the hole 53 is air, and the refractive index of air is sufficiently smaller than that of quartz-based glass. Thus, the hole assisted optical fiber has a function of returning light leaked from the core 52 by bending or the like to the core 52 again, and has a small bending loss.

### (3) Hole Structure Optical Fiber

This optical fiber has a hole group 53a of the plurality of holes 53 in the clad 60, and has a refractive index effectively lower than that of a host material (glass or the like). This structure is called a photonic crystal fiber. This structure can have a structure in which a high refractive index core having a changed refractive index does not exist, and light can be confined using a region 52a surrounded by the holes 53 as an effective core region. Compared with an optical fiber having a solid core, the photonic crystal fiber can reduce influences of absorption and scattering loss due to additives in the core, and can achieve optical characteristics that cannot be achieved by a solid optical fiber, such as reduction of bending loss and control of a nonlinear effect.

### (4) Hollow Core Optical Fiber

In this optical fiber, the core region is formed of air. The clad region has a photonic bandgap structure formed by a plurality of holes or an anti-resonance structure formed of a thin glass wire, whereby light can be confined in the core region. The optical fiber has a small nonlinear effect, and can supply a high-power or high-energy laser.

### (5) Coupling Core Type Optical Fiber

This optical fiber has a plurality of the solid cores 52 that has a high refractive index arranged close to each other in the clad 60. The optical fiber guides light between the solid cores 52 by optical wave coupling. Since the coupling core type optical fiber can disperse and transmit light by the number of cores, the power can be increased accordingly and efficient sterilization can be performed, and the coupling core type optical fiber has an advantage that fiber degradation due to ultraviolet rays can be alleviated and the life can be extended.

### (6) Solid Core Type Multi-Core Optical Fiber

This optical fiber has a plurality of the solid cores 52 that has a high refractive index arranged apart from each other in the clad 60. The optical fiber guides light in a state where an influence of optical wave coupling can be ignored by sufficiently reducing the optical wave coupling between the solid cores 52. Thus, the solid core type multi-core optical fiber has an advantage that each core can be treated as an independent waveguide.

### (7) Hole Assisted Type Multi-Core Optical Fiber

This optical fiber has a structure in which multiple hole structures and core regions of the foregoing (2) are arranged in the clad 60.

### (8) Hole Structure Type Multi-Core Optical Fiber

This optical fiber has a structure in which multiple hole structures of the foregoing (3) are arranged in the clad 60.

### (9) Hollow Core Type Multi-Core Optical Fiber

This optical fiber has a structure in which multiple hole structures of the foregoing (4) are arranged in the clad 60.

### (10) Coupling Core Type Multi-Core Optical Fiber

This optical fiber has a structure in which multiple coupling core structures of the foregoing (5) are arranged in the clad 60.

The irradiation unit 13 irradiates a predetermined irradiation target ste on which sterilization or the like is performed, with the ultraviolet light transmitted through the optical fiber 14. The irradiation unit 13 includes an optical system such as a lens designed for a wavelength of the ultraviolet light, narrows a beam of the ultraviolet light with the optical system, and irradiates the irradiation target ste with the ultraviolet light with pinpoint accuracy. In the present embodiment, it is assumed that an irradiation direction of the ultraviolet light (a direction in which an irradiation target can be irradiated with the ultraviolet light) or a beam diameter is adjusted and fixed in advance by an operator or the like.

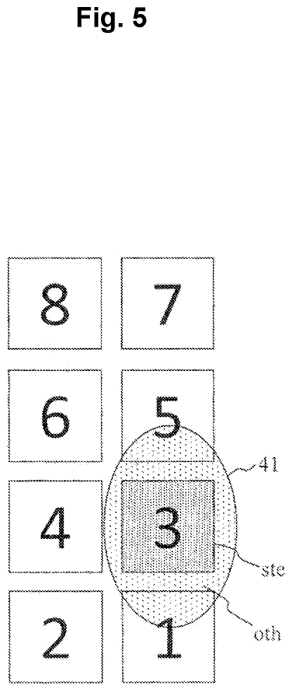

Note that “irradiate with pinpoint accuracy” means the following (see FIG. 5). FIG. 5 is a diagram in which one of destination floor buttons (irradiation target ste) of an elevator is irradiated with a beam of ultraviolet light. When a wall or the like is irradiated with a beam of ultraviolet light, a beam spot 41 of the beam is a circle or an ellipse. The irradiation target ste is included in the circular or elliptical beam spot 41, and an area of a portion both irradiated with the ultraviolet light other than the irradiation target ste is made as small as possible.

The irradiation target ste is touched by a person. The irradiation target ste is, for example, a destination floor button in an elevator hall or an elevator, a strap in a bus or a train, or the like.

The sensing unit 16 monitors a state of the irradiation target ste and transmits monitoring information Info on the state to the irradiation control unit 15. For example, the sensing unit 16 is a monitoring camera, and acquires monitoring information Info indicating when and which elevator button is touched by a person and whether there is a person in the elevator by image recognition processing, and transmits the monitoring information Info to the irradiation control unit 15. In addition, the sensing unit 16 may be a sensor arranged in each irradiation target ste, and detect that a person touches the sensor. Note that a communication path 17a from the sensing unit 16 to the irradiation control unit 15 may be wired or wireless.

On the basis of the monitoring information Info from the sensing unit 16, the irradiation control unit 15 determines a time and a place in which irradiation of the ultraviolet light is necessary. For example, the irradiation control unit 15 determines that a button touched by a person needs to be irradiated with the ultraviolet light for 10 seconds. The irradiation control unit 15 also determines the presence or absence of a person in the vicinity of the irradiation target ste from the monitoring information Info. The irradiation control unit 15 gives an instruction 17b to output the ultraviolet light to the ultraviolet light source unit 11 on the basis of the determination result. Specifically, the irradiation control unit 15 determines which destination floor button in the elevator is touched by a person on the basis of the monitoring information Info, and further confirms that there is no person in the elevator, and then instructs the ultraviolet light source 11 capable of irradiating the button with the ultraviolet light to output the ultraviolet light. Here, the intensity of the ultraviolet light and the irradiation time may be appropriately changed.

With such a configuration, the ultraviolet light irradiation system 301 can perform sterilization or the like on a button or the like of an elevator used by an unspecified number of people by pinpoint ultraviolet light irradiation, and can avoid material degradation of other portions.

### Second Embodiment

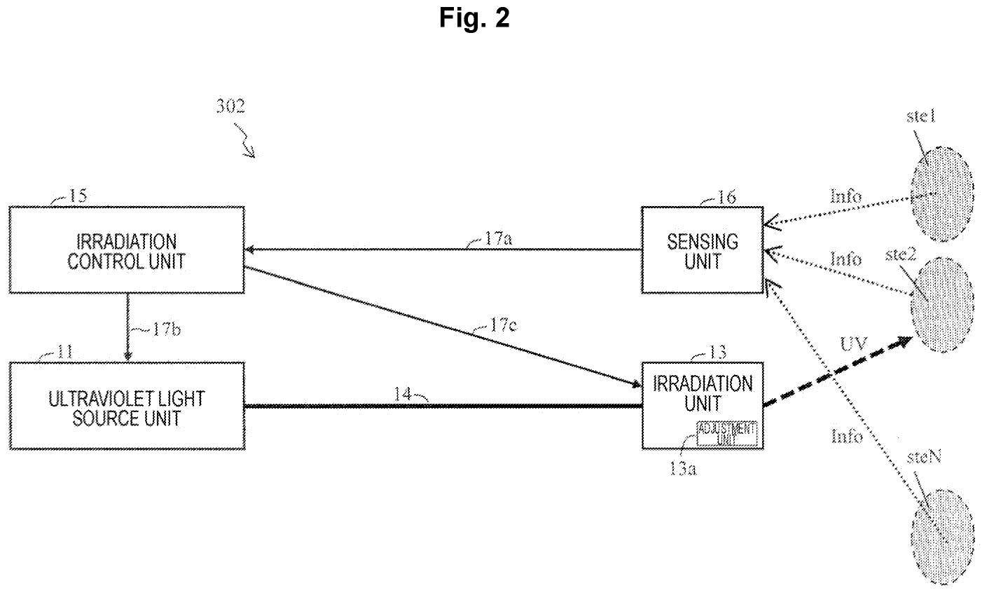

FIG. 2 is a diagram illustrating an ultraviolet light irradiation system 302 of the present embodiment. The ultraviolet light irradiation system 302 further has the following functions with respect to the ultraviolet light irradiation system 301 of FIG. 1. The irradiation unit 13 includes an adjustment unit 13a that adjusts the irradiation direction of the ultraviolet light. The monitoring information Info also includes information on a position of the irradiation target ste touched by a person. The irradiation control unit 15 instructs the adjustment unit 13a on the irradiation direction of the ultraviolet light on the basis of the monitoring information Info.

In the present embodiment, only portions different from the ultraviolet light irradiation system 301 will be described.

The adjustment unit 13a is an actuator and adjusts the irradiation direction of the ultraviolet light.

The sensing unit 16 also detects information on the position of the irradiation target ste touched by a person (for example, in the case of FIG. 5, information indicating which destination floor button is touched by a person), and includes the information in the monitoring information Info.

On the basis of the information on the position of the irradiation target ste touched by the person in the monitoring information Info, the irradiation control unit 15 instructs the adjustment unit 13a of the irradiation unit 13 to adjust the beam of the ultraviolet light so that the beam is directed to the irradiation target ste touched by the person. Specifically, the irradiation control unit 15 determines which destination floor button in the elevator is touched by a person on the basis of the monitoring information Info, and further confirms that there is no person in the elevator, and then instructs the adjustment unit 13a to perform beam adjustment so that the button can be irradiated with the ultraviolet light. Note that a communication path 17c from the irradiation control unit 15 to the adjustment unit 13a may be wired or wireless.

With such a configuration, the ultraviolet light irradiation system 302 can perform sterilization or the like on a button or the like of an elevator used by an unspecified number of people by pinpoint ultraviolet light irradiation, and can avoid material degradation of other portions. In addition, the ultraviolet light irradiation system 302 can perform sterilization or the like on a large number of irradiation targets ste with one ultraviolet light source unit, and is economical.

### Third Embodiment

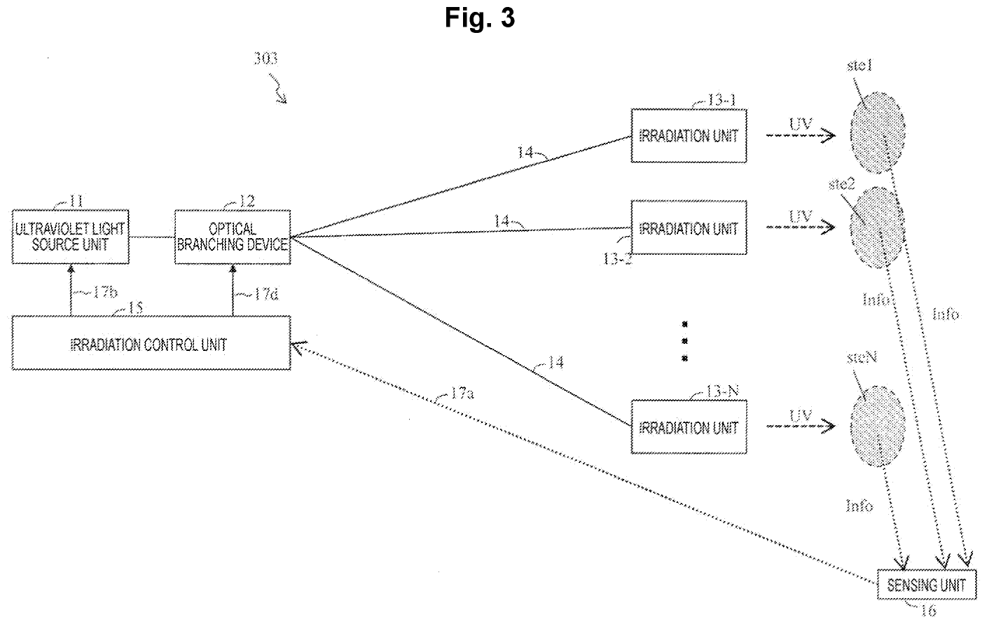

FIG. 3 is a diagram illustrating an ultraviolet light irradiation system 303 of the present embodiment. The ultraviolet light irradiation system 303 further has the following functions with respect to the ultraviolet light irradiation system 301 of FIG. 1.

There is a plurality of the irradiation units 13. An optical branching device 12 that splits the ultraviolet light to each of the irradiation units 13 is further included.

In the present embodiment, only portions different from the ultraviolet light irradiation system 301 will be described.

In the present embodiment, the number of the irradiation units 13 is N (N is a natural number greater than or equal to 2). The irradiation units 13 are adjusted to irradiate the respective irradiation targets ste different from each other (for example, in the case of FIG. 5, respective destination floor buttons) with ultraviolet light.

The optical branching device 12 distributes the ultraviolet light from the ultraviolet light source unit 11 to each irradiation unit 13. The optical branching device 12 is, for example, an optical splitter or an optical switch.

The sensing unit 16 also detects information on the position of the irradiation target ste touched by a person (for example, in the case of FIG. 5, information indicating which destination floor button is touched by a person), and includes the information in the monitoring information Info.

On the basis of the information on the position of the irradiation target ste touched by the person in the monitoring information Info, the irradiation control unit 15 gives an instruction 17d to switch paths to the optical branching device 12 so that the irradiation target ste touched by the person is irradiated with the ultraviolet light. Specifically, the irradiation control unit 15 determines which destination floor button in the elevator is touched by a person on the basis of the monitoring information Info, and further confirms that there is no person in the elevator, and then instructs the optical branching device 12 to switch optical paths so that the button can be irradiated with the ultraviolet light.

Note that, in a case where the optical branching device 12 is an optical splitter, path switching cannot be performed unlike an optical switch. In this case, the irradiation control unit 15 confirms that there is no person in the elevator on the basis of the monitoring information Info, and then instructs the ultraviolet light source unit 11 to output the ultraviolet light.

With such a configuration, the ultraviolet light irradiation system 303 can perform sterilization or the like on a button or the like of an elevator used by an unspecified number of people by pinpoint ultraviolet light irradiation, and can avoid material degradation of other portions. In addition, the ultraviolet light irradiation system 303 can perform sterilization or the like on a large number of irradiation targets ste with one ultraviolet light source unit, and is economical.

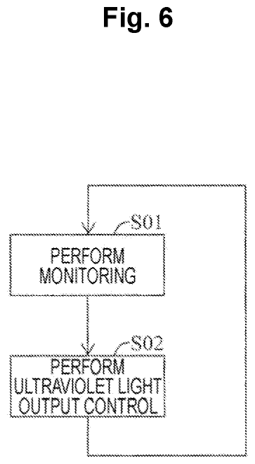

FIG. 6 is a flowchart for explaining a control method for controlling operation of the ultraviolet light irradiation system (301 to 303). The present control method is a control method for the above-described ultraviolet light irradiation system,
the control method including:

outputting the state of the desired portion ste as monitoring information by the sensing unit 16 (step S01); and
controlling output of the ultraviolet light from the ultraviolet light source unit 11 on the basis of the monitoring information (S02).

The ultraviolet light irradiation system (301 to 303) may repeat steps S01 and S02.

### REFERENCE SIGNS LIST

11 ultraviolet light source unit
12 optical branching device
13 irradiation unit
13a adjustment unit
14 optical fiber
15 irradiation control unit
16 sensing unit
41 beam spot
52 solid core
52a region
53 hole
53a hole group
60 clad
301 to 303 ultraviolet light irradiation system
ste irradiation target (target to be irradiated with ultraviolet light)

1. An ultraviolet light irradiation system comprising:
one ultraviolet light source unit that generates ultraviolet light;
an irradiation unit that irradiates a desired portion with the ultraviolet light in a limited manner;
an optical fiber that propagates the ultraviolet light from the ultraviolet light source unit to the irradiation unit;
a sensing unit that monitors a state of the desired portion and outputs the state as monitoring information; and
an irradiation control unit that controls output of the ultraviolet light from the ultraviolet light source unit on a basis of the monitoring information.

2. The ultraviolet light irradiation system according to claim 1, wherein the monitoring information is information indicating that the desired portion is touched by a person.

3. The ultraviolet light irradiation system according to claim 2, wherein
the irradiation unit includes an adjustment unit that adjusts an irradiation direction of the ultraviolet light,
the monitoring information also includes information on a position of the desired portion touched by a person, and
the irradiation control unit instructs the adjustment unit on the irradiation direction of the ultraviolet light on a basis of the monitoring information.

4. The ultraviolet light irradiation system according to claim 1, wherein
the irradiation unit includes a plurality of the irradiation units, and
an optical branching device that splits the ultraviolet light to each of the irradiation units is further included.

5. The ultraviolet light irradiation system according to claim 1, wherein
the optical fiber is any one of a solid core optical fiber, a hole assisted optical fiber, a hole structure optical fiber, a hollow core optical fiber, a coupling core type optical fiber, a solid core type multi-core optical fiber, a hole assisted type multi-core optical fiber, a hole structure type multi-core optical fiber, a hollow core type multi-core optical fiber, and a coupling core type multi-core optical fiber.

6. A control method for an ultraviolet light irradiation system including:
one ultraviolet light source unit that generates ultraviolet light;
an irradiation unit that irradiates a desired portion with the ultraviolet light in a limited manner;
an optical fiber that propagates the ultraviolet light from the ultraviolet light source unit to the irradiation unit; and
a sensing unit that monitors a state of the desired portion,
the control method comprising:
outputting the state of the desired portion as monitoring information by the sensing unit; and
controlling output of the ultraviolet light from the ultraviolet light source unit on a basis of the monitoring information.
